(19) United States
(12) Patent Application Publication
(10) Pub. No.: US 2015/0230743 A1
(43) Pub. Date: Aug. 20, 2015

(54) SENSOR CONFIGURATIONS FOR ANATOMICAL VARIATIONS

(71) Applicant: COVIDIEN LP, MANSFIELD, MA (US)

(72) Inventors: Paulo E.X. Silveira, Boulder, CO (US); Sarah Hayman, Boulder, CO (US); Jill Klomhaus Anderson, Longmont, CO (US); Paul S. Addison, Edinburgh (GB); James N. Watson, Dunfermline (GB)

(21) Appl. No.: 14/593,602

(22) Filed: Jan. 9, 2015

Related U.S. Application Data
(60) Provisional application No. 61/940,671, filed on Feb. 17, 2014.

(51) Int. Cl. A61B 5/1455; A61B 5/00

(52) U.S. Cl. CPC A61B 5/14552; A61B 5/7282; A61B 5/742; A61B 5/746

(57) ABSTRACT

A medical sensor includes a first set of optical components configured to obtain a first set of signals for determining a first regional oxygen saturation measurement. The first set of optical components includes a first emitter, a first detector separated from the first emitter by a first distance along a first axis, and a second detector separated from the first emitter by a second distance along the first axis, wherein the second distance is greater than the first distance. The sensor also includes a second set of optical components configured to obtain a second set of signals for determining a second regional oxygen saturation measurement. The second set of optical components includes a second emitter and a third detector separated from the second emitter by a third distance along a second axis, different from the first axis.

### CROSS REFERENCE TO RELATED APPLICATIONS

This application claims the benefit of U.S. Provisional Patent Application No. 61/940,671, filed Feb. 17, 2014, entitled “Sensor Configurations for Anatomical Variations,” which is incorporated by reference herein in its entirety.

### BACKGROUND

The present disclosure relates generally to medical devices and, more particularly, to medical sensors and systems for determining physiological parameters, such as plethysmographically-determined parameters.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of devices have been developed for non-invasively monitoring physiological characteristics of patients. For example, an oximetry sensor system may non-invasively measure and monitor various blood flow characteristics of a patient, such as a blood oxygen saturation of hemoglobin in arterial blood, a volume of individual blood pulsations supplying the tissue, and/or the rate of blood pulsations corresponding to each heart beat of a patient. During operation, the oximeter sensor emits light and photoelectrically senses the absorption and/or scattering of the light after passage through the perfused tissue. A photoplethysmographic waveform, which corresponds to the cyclic attenuation of optical energy through the patient's tissue, may be generated from the detected light. Additionally, one or more physiological characteristics may be calculated based upon the amount of light absorbed or scattered. More specifically, the light passed through the tissue may be selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

For example, a regional saturation sensor may be applied to the patient's head or other body location to monitor the patient. Such sensors generally include one or more emitters that emit the light and one or more detectors that detect the light. The light detected may be used to calculate one or more of the above physiological characteristics based upon the absorption or scattering of light. The one or more emitters and the one or more detectors may be coupled to an oximeter sensor that couples to an oximeter monitor. Attaining accurate and reliable data may be difficult with typical oximeter sensor configurations, which only monitor a relatively small area of the patient's tissue, for example. Additionally, attaining accurate and reliable data may be difficult with typical oximeter sensor configurations due to lack of redundancy and/or symmetry in measurements, and/or due to the limited availability of light penetration depths and tissue monitoring volume provided by such typical sensor configurations.

### BRIEF DESCRIPTION OF THE DRAWINGS

Advantages of the disclosed techniques may become apparent upon reading the following detailed description and upon reference to the drawings in which:

FIG. 1 is a front perspective view of an embodiment of a monitoring system configured to be used with a sensor for regional saturation;

FIG. 2 is a block diagram of the monitoring system of FIG. 1;

FIG. 3 is a bottom view of an embodiment of a sensor configured to be used in conjunction with the monitoring system of FIG. 1 to obtain two regional saturation measurements using two emitters and three detectors;

FIG. 4 is a side view of the sensor of FIG. 3 applied to a patient, in accordance with an embodiment;

FIG. 5 is a bottom view of an embodiment of a sensor configured to obtain four regional saturation measurements using two emitters and six detectors;

FIG. 6 is a bottom view of an embodiment of a sensor configured to obtain four regional saturation measurements using four emitters and five detectors;

FIG. 7 is a bottom view of an embodiment of a sensor configured to obtain six regional saturation measurements using six emitters and three detectors;

FIG. 8 is a bottom view of an embodiment of a sensor configured to obtain two regional saturation measurements using two emitters and two detectors and having additional emitters configured to monitor tissue uniformity;

FIG. 9 is a bottom view of an embodiment of a sensor configured to obtain two regional saturation measurements using two emitters configured to emit alternating wavelengths of light and two detectors and having additional emitters configured to monitor tissue uniformity; and

FIG. 10 is a bottom view of an embodiment of a sensor configured to obtain two regional saturation measurements using emitters and detectors arranged linearly in an alternating pattern;

FIG. 11 is a bottom view of an embodiment of a sensor configured to obtain two regional saturation measurements using two sensing units, each sensing unit having three detectors; and

FIG. 12 is a bottom view of an embodiment of a sensor configured to obtain four regional saturation measurements using one emitter and four sets of detectors arranged about the emitter.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

The present embodiments relate generally to sensor designs or configurations for non-invasive patient monitoring using near-infrared spectroscopy. Such sensors may include optical elements configured for regional oxygen saturation monitoring or photoplethysmography. For example, the sensors disclosed herein may include one or more emitters and one or more detectors for determining the blood oxygen saturation in a particular region of a patient's body, such as a cerebral or somatic region. Further, the sensors disclosed herein may include features to facilitate sensing across multiple tissue levels. In particular embodiments, the disclosed sensors may provide certain advantages over traditional sensors. For example, the sensors described herein may provide symmetry and/or redundancy, for more accurate and/or reliable and/or robust regional oxygen saturation measurements. Additionally, the sensors described herein may be configured to monitor a relatively large two-dimensional area and a relatively large volume of tissue as compared to traditional regional saturation sensors, for example, resulting in increased robustness to variations in sensor placement. Thus, the regional saturation measurements obtained may be less sensitive to non-uniformities in the tissue or local changes in the tissue, and may therefore provide more accurate, reliable, and robust regional saturation measurements for the region or organ of interest. Additionally, although the present embodiments are discussed in the context of regional oxygen saturation, it should be understood that other types of sensors or patient monitoring systems (e.g., those used for measuring arterial oxygen saturation, water fraction, hematocrit, or bispectral index) may benefit from the configurations and/or techniques disclosed herein.

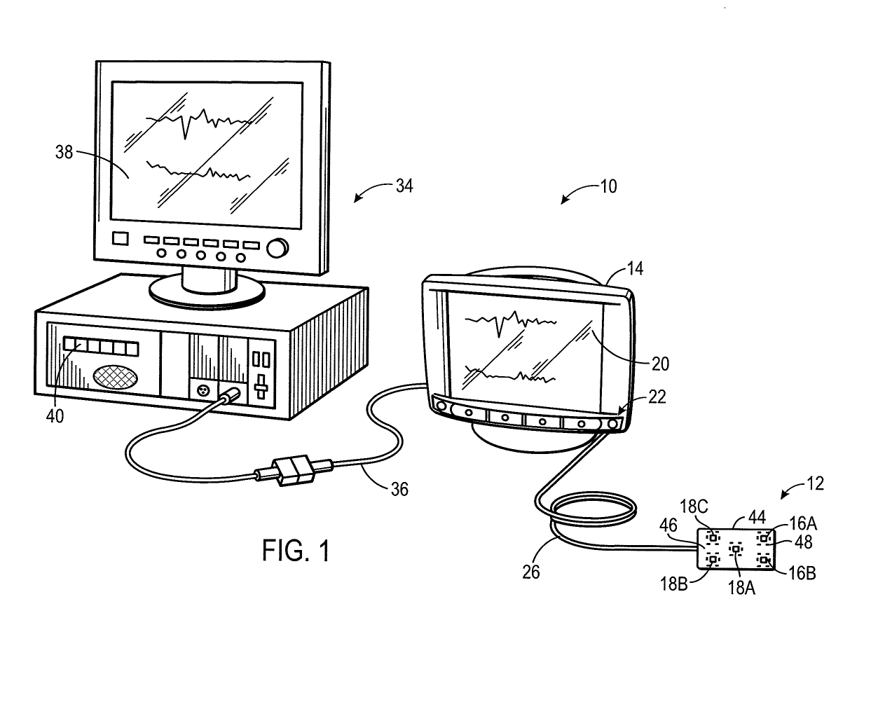

With the foregoing in mind, FIG. 1 depicts an embodiment of a patient monitoring system 10 that may be used in conjunction with a medical sensor 12. In certain embodiments, the features of the sensor 12 as provided herein may be incorporated into sensors for use at a wide variety of tissue locations, such as a head, back, a stomach, a heel, an arm, a leg, an ear, or any other appropriate measurement site. In addition, although the embodiment of the patient monitoring system 10 illustrated in FIG. 1 relates to photoplethysmography or regional oximetry, the system 10 may be configured to obtain a variety of medical measurements with any suitable medical sensor. For example, the system 10 may, additionally be configured to determine blood pressure, respiration rate, patient electroencephalography (e.g., a bispectral index), or any other desired physiological parameter.

As noted, the system 10 includes the sensor 12 that is communicatively coupled to a patient monitor 14. Although only one sensor 12 is shown coupled to the monitor 14 in FIG. 1, in other embodiments, two, three, four, or more sensors 12 may be coupled to the monitor 14. The sensor 12 may include one or more emitters 16 and one or more detectors 18. In the particular embodiment illustrated, the sensor 12 includes two emitters 16A, 16B and three detectors 18A, 18B, 18C. Each of the emitters 16 and the detectors 18 of the sensor 12 are coupled to the monitor 14 via a cable 26 coupled to the monitor 14. The cable 26 may interface directly with the sensor 12 and may include a plurality of conductors (e.g., wires).

The monitor 14 includes a monitor display 20 configured to display information regarding the physiological parameters monitored by the sensor 12, information about the system 10, and/or alarm indications, for example. The monitor 14 may include various input components 22, such as knobs, switches, keys and keypads, buttons, etc., to facilitate operation and configuration of the monitor 14. As discussed in more detail below, the monitor 14 also includes a processor that may be used to execute code, such as code for implementing any of the various monitoring functionalities enabled by the sensor 12. For example, the monitor 14 may be configured to process signals generated by the detectors 18 to estimate the amount of oxygenated hemoglobin in a monitored region of the patient.

In some embodiments, the monitor 14 may be coupled to a multi-parameter patient monitor 34 via a cable 36 connected to a sensor input port. In addition to the monitor 14, or alternatively, the multi-parameter patient monitor 34 may be configured to calculate physiological parameters and to provide a central display 38 for the visualization of information from the monitor 14 and from other medical monitoring devices or systems. The multi-parameter monitor 34 includes a processor that may be configured to execute code. The multi-parameter monitor 34 may also include various input components 40, such as knobs, switches, keys and keypads, buttons, etc., to provide for operation and configuration of the multi-parameter monitor 34. In addition, the monitor 14 and/or the multi-parameter monitor 34 may be connected to a network to enable the sharing of information with servers or other workstations.

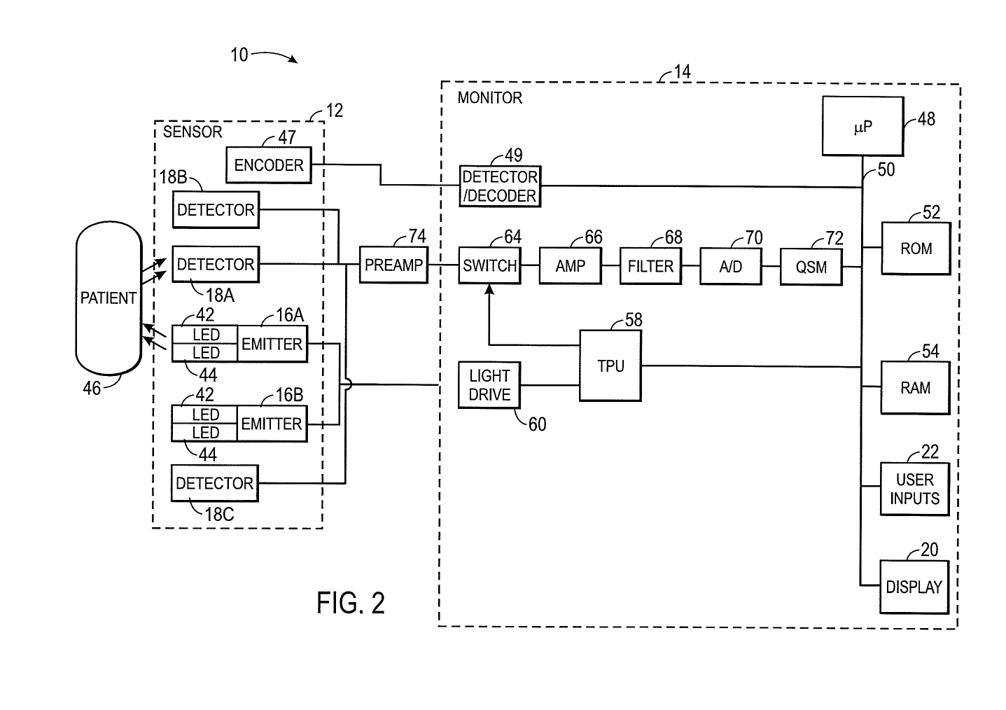

In certain embodiments, the sensor 12 may be a wireless sensor 12. Accordingly, the sensor 12 may establish a wireless communication with the patient monitor 14 and/or the multi-parameter patient monitor 34 using any suitable wireless standard. The patient monitor 14 may establish a wireless communication with the multi-parameter patient monitor 34. By way of example, the wireless module may be capable of communicating using one or more of the ZigBee standard, WirelessHART standard, Bluetooth standard, IEEE 802.11x standards, or MiWi standard. In certain embodiments, a pre-amplifier (see FIG. 2) may be provided between the sensor 12 and the monitor 14 to convert analog signals into digital signals, computer parameters, and/or communicate the same robustly to the monitor 14, for example.

The sensor 12, illustrated as operatively connected to the monitor 14, may include a sensor body 44 (e.g., a substrate) that houses and/or supports the one or more emitters 16 for emitting light at certain wavelengths into a tissue of a patient and the one or more detectors 18 for detecting the light after it is reflected and/or absorbed by the blood and/or tissue of the patient. The sensor body 44 may be formed from any suitable material, including rigid or conformable materials, such as fabric, paper, rubber or elastomeric compositions (including acrylic elastomers, polyimide, silicones, silicone rubber, celluloid, PDMS elastomer, polyurethane, polypropylene, acrylics, nitrile, PVC films, acetates, or latex).

A patient-contacting surface 46 of the sensor 12 may be an adhesive layer, in some embodiments. Thus, the patient-contacting surface 46 may include any adhesive material suitable for integration into medical devices (e.g., a hypoallergenic adhesive material). In some embodiments, the adhesive material may be substantially transparent with respect to the wavelengths of light used for the oximetry measurements performed by the sensor 12. In other embodiments where the patient-contacting adhesive layer 46 is not transparent with respect to the wavelengths of light used for the oximetry measurements, the patient-contacting adhesive layer 46 may include optical windows 48 (e.g., openings), illustrated as dashed lines, corresponding to the respective positions of the one or more emitters 16 and the one or more detectors 18. By way of example, the patient-contacting surface 46 may include an acrylic adhesive, a silicon-based adhesive, or a hydrocolloid adhesive. Generally, hydrocolloid adhesives may provide enhanced comfort for the patient and avoid damage to the patient's skin when the sensor 12 is removed or repositioned. A release liner may also be provided to prevent the inadvertent attachment of the patient-contacting surface 46 to a surface before the intended use of the sensor 12. The release liner may include any liner having a release material suitable for use with the patient-contacting surface 46, such as a coated release paper or a release plastic film. Example release materials include polyolefins (e.g., polypropylene, high- and low-density polyethylene), polyesters (e.g., biaxially-oriented polyethylene terephthalate), polyvinyl alcohol, polystyrene, or the like.

As provided herein, the sensor 12 may be configured to perform regional oximetry. In regional oximetry, by comparing the relative intensities of light received at two or more detectors 18, it is possible to estimate the blood oxygen saturation of hemoglobin in a region of a body. Whereas pulse oximetry measures blood oxygen based on changes in the volume of blood due to pulsing tissue (e.g., arteries), regional oximetry typically examines blood oxygen saturation within the venous, arterial, and capillary systems within a region of a patient. For example, a regional oximeter system 10 may include the sensor 12 configured to be placed on a patient's forehead and may be used to calculate the oxygen saturation of a patient's blood within the venous, arterial, and capillary systems of a region underlying the patient's forehead (e.g. in the cerebral cortex). As illustrated in FIG. 1, the sensor 12 may include two emitters 16A, 16B (e.g., a first emitter and a second emitter) and three detectors 18: one detector 18A (e.g., a first detector) that is relatively “close” to the emitters 16A, 16B and two detectors 18B, 18C (e.g., a second detector and a third detector) that are relatively “far” from the two emitters 16A, 16B. Light intensity of one or more wavelengths may be received at the “close” and the “far” detectors 18A, 18B, 18C. Thus, the close detector 18A may receive a first portion of light and the far detectors 18B, 18C may receive a second portion of light. Each of the detectors 18 may generate signals indicative of their respective portions of light, and the resulting signals may be compared to arrive at a regional saturation value that pertains to additional tissue through which the light received at the “far” detector 18B, 18C passed (tissue in addition to the tissue through which the light received by the close detector 18A passed, e.g., the brain tissue) when it was transmitted through a region of a patient (e.g., a patient's cranium).

In certain embodiments, light emitted by the first emitter 16A may be received at the first detector 18A and the second detector 18B, and the signals generated based on the light received by the first detector 18A and the second detector 18B may be utilized to generate a first oxygen saturation value. Additionally or alternatively, light emitted by the second emitter 16B may be received at the first detector 18A and the third detector 18C, and the signals generated based on the light received by the first detector 18A and the third detector 18C may be compared to generate a second oxygen saturation value. For example, the first and the second regional oxygen saturation values may be determined via the following equations:

R=ln([I₁(λ₁)]/[I₁(λ₂)/I₂(λ₂)]) (1)

and

rSO₂=S=R(AMb(r₂−r₁)[c])⁻¹−(B/A) (2)

In the above equations, I₁, I₂, r₁, and r₂ are the signal intensities and distances between the near detector 18A and the far detectors 18B, 18C, respectively. λ₁ and λ₂ refer to the two wavelengths of light emitted by each of the emitters 16A, 16B, and A and B are constants related to the extinction coefficients of oxygenated and reduced hemoglobin. In certain embodiments, the second wavelength (λ₂) may be an isobestic point (e.g., 804 nm). As discussed in more detail below, in some embodiments, the wavelengths of light emitted by one emitter (e.g., emitter 16A) may be different and/or alternate with the wavelengths of light emitted by another emitter (e.g., emitter 16B). [c] is the combined concentration of all hemoglobin, additionally factored by the concentration of blood in the tissue. Mb is an empirically determined constant related to the mean path length of light through the subdermal tissue. The foregoing is merely illustrative and any suitable equations and/or processing techniques may be used to calculate regional oximetry values, in accordance with the present embodiments. Additionally, surface data from the skin and skull may be subtracted out to produce a regional oxygen saturation (rSO₂) value for deeper tissues. Other methods to calculate regional blood oxygen saturation, such as those provided in U.S. Pat. Nos. 5,139,025 and 5,217,013 or U.S. Patent Publication No. 2011/0112387, filed Nov. 12, 2009, the disclosures of which are incorporated by reference in their entirety herein for all purposes, may be employed.

Turning to FIG. 2, a block diagram of an embodiment of the medical system 10 of FIG. 1 is illustrated. The sensor 12 may include optical components in the form of one or more emitters 16 and one or more detectors 18. As shown, the sensor 12 includes two emitters 16A, 16B and three detectors 18A, 18B, 18C. The one or more emitters 16 and the one or more detectors 18 may be arranged in a reflectance or transmission-type configuration with respect to one another. However, in embodiments in which the sensor 12 is configured for use on a patient's forehead, the one or more emitters 16 and the one or more detectors 18 may be in a reflectance configuration.

The one or more emitters 16 may be a light emitting diode, superluminescent light emitting diode, a laser diode, or a vertical cavity surface emitting laser (VCSEL). The one or more emitters 16 and the one or more detectors 18 may include optical fiber sensing elements. The one or more emitters 16 may include a broadband or “white light” source, in which case the one or more detectors 18 could include any of a variety of elements for selecting specific wavelengths, such as reflective or refractive elements or interferometers. Alternatively, the sensor 12 may sense light detected from the tissue is at a different wavelength from the light emitted into the tissue. Such sensors may be adapted to sense fluorescence, phosphorescence, Raman scattering, Rayleigh scattering and multi-photon events, or photoacoustic effects.

In one embodiment, the one or more emitters 16 may be configured for use in a regional saturation technique. To that end, each of the one or more emitters 16 may include two light emitting diodes (LEDs) 42 and 44 (e.g., a first LED and a second LED) that are configured to emit at least two wavelengths of light, e.g., red or near infrared light. In one embodiment, the LEDs 42 and 44 emit light in the range of 600 nm to about 1000 nm. In a particular embodiment, the first LED 42 is configured to emit light at about 730 nm and the second LED 44 is configured to emit light at about 810 nm. In some embodiments, as discussed in more detail below, one or more emitters 16 may be configured to provide wavelengths of light different from other emitters 16 of the sensor 12. For example, the LEDs 42 and 44 of the first emitter 16A may provide two wavelengths of light (e.g., about 724 nm and 812 nm, respectively), and the LEDs 42 and 44 of the second emitter 16B may provide two wavelengths of light (e.g., about 770 nm and 850 nm, respectively) different from the two wavelengths of light provided by the first emitter 16A. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In any suitable configuration of the sensor 12, the one or more detectors 18 may each be an array of detector elements configured to detect light at various intensities and wavelengths. In one embodiment, light enters the one or more detectors 18 after passing through the tissue of the patient 46. In another embodiment, light emitted from the one or more emitters 16 may be scattered by multiple elements in the patent's tissue to be reflected back and enter the one or more detectors 18. The one or more detectors 18 may convert the received light at a given intensity, which may be directly related to the absorbance and/or scattering of light in the tissue of the patient 46, into an electrical signal. That is, when more light at a certain wavelength is absorbed, less light of that wavelength is typically received from the tissue by the one or more detectors 18, and when more light at a certain wavelength is scattered and reflected back, more light of that wavelength is typically received from the tissue by the one or more detectors 18. After converting the received light to an electrical signal, the one or more detectors 18 may send the signal to the monitor 14, where physiological characteristics may be calculated based at least in part on the absorption and/or scattering of light by the tissue of the patient 46.

In certain embodiments, the medical sensor 12 may also include an encoder 47 that may provide signals indicative of the wavelength of one or more light sources of the one or more emitters 16, which may allow for selection of appropriate calibration coefficients for calculating a physical parameter such as blood oxygen saturation. The encoder 47 may, for instance, include a coded resistor, an electrically erasable programmable read only memory (EEPROM), or other coding device (such as a capacitor, inductor, programmable read only memory (PROM), RFID, parallel resident currents, or a colorimetric indicator) that may provide a signal to a microprocessor 48 related to the characteristics of the medical sensor 12 to enable the microprocessor 48 to determine the appropriate calibration characteristics of the medical sensor 12. The encoder 47 may also include information related to the number of emitters 16, the number of detectors 18, and/or the configuration of and/or spacing between the emitters 16 and detectors 18, for example. Further, the encoder 47 may include encryption coding that prevents a disposable part of the medical sensor 12 from being recognized by a microprocessor 48 unable to decode the encryption. For example, a detector/decoder 49 may translate information from the encoder 47 before it can be properly handled by the processor 48. In some embodiments, the encoder 47 and/or the detector/decoder 49 may not be present.

Signals from the one or more detectors 18 and/or the encoder 47 may be transmitted to the monitor 14. The monitor 14 may include one or more processors 48 coupled to an internal bus 50. Also connected to the bus 50 may be a ROM memory 52, a RAM memory 54, and the display 20. A time processing unit (TPU) 58 may provide timing control signals to light drive circuitry 60, which controls when each of the one or more emitters 16 are activated, and if multiple light sources are used, the multiplexed timing for the different light sources. It is envisioned that the emitters 16 may be controlled via time division multiplexing of the light sources. Thus, in some embodiments, the light from each of the one or more emitters 16 may be emitted in staggered, or alternating, manner TPU 58 may also control the gating-in of signals from the one or more detectors 18 through a switching circuit 64. These signals are sampled at the proper time, depending at least in part upon which of the multiple light sources is activated, if multiple light sources are used. In certain embodiments, multiple emitters 16 may emit different wavelengths of light simultaneously, and the signals received by the one or more detectors 18 may be separated (e.g., filtered) based on wavelength or a modulation frequency, for example, to enable determination of one or more regional saturation measurements using the appropriate signals. The received signal(s) from the one or more detectors 18 may be passed through an amplifier 66, a low pass filter 68, and an analog-to-digital converter 70 for amplifying, filtering, and digitizing the electrical signals from the ear sensor 12. The digital data may then be stored in a queued serial module (QSM) 72, for later downloading to RAM 54 as QSM 72 fills up. In an embodiment, there may be multiple parallel paths for separate amplifiers, filters, and A/D converters for multiple light wavelengths or spectra received.

In an embodiment, based at least in part upon the received signals corresponding to the light received by the detector 18, the processor 48 may calculate the oxygen saturation using various algorithms, such as the algorithms set forth above with respect to FIG. 1. These algorithms may use coefficients, which may be empirically determined For example, algorithms relating to the distance between the one or more emitters 16 and the one or more detectors 18 may be stored in the encoder 47, the ROM memory 52, or any suitable location, and accessed and operated according to processor 48 instructions.

Furthermore, one or more functions of the monitor 14 may be implemented by a pre-amplifier 74. In some embodiments, the pre-amplifier 74 may be configured to carry out all of the functions of the monitor 14, except for providing the display 20 and the user inputs 22. In certain embodiments, one or more functions of the monitor 14 may be implemented directly in the sensor 12. For example, in some embodiments, the sensor 12 may include one or more processing components capable of calculating the physiological characteristics from the signals obtained from the patient 46. In accordance with the present techniques, the sensor 12 may be configured to provide desired contact between the patient 46 and the detector 18, and/or the emitter 16. The sensor 12 may have varying levels of processing power, and may output data in various stages to the monitor 14, either wirelessly or via the cable 26. For example, in some embodiments, the data output to the monitor 14 may be analog signals, such as detected light signals (e.g., oximetry signals or regional saturation signals), or processed data.

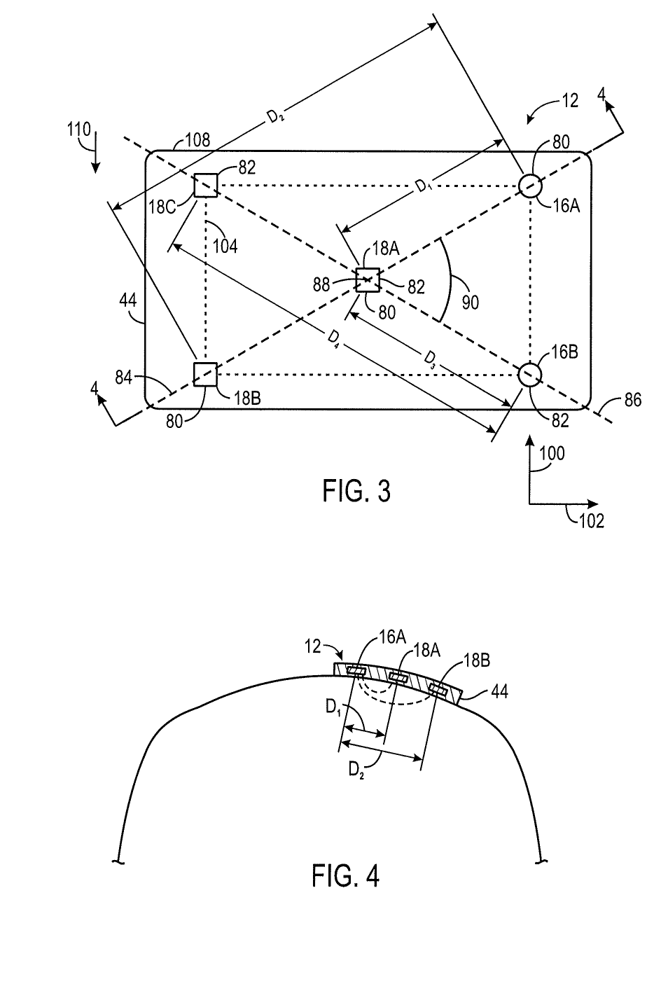

In some embodiments, the sensor 12 may include one or more emitters 16 and/or one or more detectors 18 configured to provide multiple regional saturation measurements. Such configurations may be utilized to monitor regional saturation across a large surface area of the patient's tissue and/or to provide useful comparative measurements and/or redundancy, for example. FIG. 3 is an embodiment of the sensor 12 that includes multiple optical elements (e.g., one or more emitters 16 and one or more detectors 18) arranged on the sensor body 44 to facilitate patient monitoring. As shown, the sensor 12 includes two emitters 16A, 16B and three detectors 18A, 18B, 18C arranged in the configuration shown in FIG. 1. In the illustrated embodiment, the first emitter 16A is configured to be used with the first detector 18A and the second detector 18B to obtain a first regional oxygen saturation measurement when the sensor 12 is applied to the patient. Thus, the first detector 18A and the second detector 18B form a first set of detectors, and the first emitter 16A and the first set of detectors (i.e., the first detector 18A and the second detector 18B) form a first sensing unit 80 (e.g., a set of optical components or a regional saturation sensing unit) for obtaining the first regional saturation measurement. As depicted, the sensor 12 also includes the second emitter 16B configured to be used with the first detector 18A and the third detector 18C to obtain a second regional oxygen saturation measurement when the sensor 12 is applied to the patient. Thus, the first detector 18A and the third detector 18C form a second set of detectors, and the second emitter 16B and the second set of detectors (i.e., the first detector 18A and the third detector 18C) form a second sensing unit 82 for obtaining the second regional oxygen saturation measurement. Although only two sensing units 80, 82 are depicted in FIG. 3, it should understood that any suitable number of sensing units may be provided to obtain any desired number of regional oxygen saturation values.

In the depicted configuration, the emitter 16A of the first sensing unit 80 is a first distance D₁ from the first detector 18A and a second distance D₂ from the second detector 18B. As shown, the distance D₁ is less than D₂. In addition, the second emitter 16B of the second sensing unit 82 is positioned a third distance D₃ from the first detector 18A and a fourth distance D₄ from the second detector 18C. As shown, the distance D₃ is less than D₄. As discussed in more detail below, the optical components of the sensing units 80, 82 may be arranged symmetrically, and thus, in certain embodiments, the distance D₁ may be equal to D₃ and/or the distance D₂ may be equal to D₄. Such a configuration (e.g., symmetrical and/or equal distances as noted above) may provide redundant measurements, and the redundancy can be used to increase the accuracy and/or robustness of the measurement. The accuracy can be increased, for example, by averaging the measurements from each of the sensing units 80, 82, for example. The robustness can be increased, for example, by rejecting the measurement that presents the highest amount of noise (e.g., estimated by the standard deviation of the signal), or by rejecting that measurement which deviates the most from the median value of combined measurements, as discussed in more detail below.

With reference to FIG. 4, which shows a side-cross sectional view of the sensor 12 of FIG. 3 taken along line 4-4, the emitter-detector spacing at the first distance D₁ represents a shallower optical path and the emitter-detector spacing at the second distance D₂, greater than the first distance D₁, represents a deeper optical path for tissue penetration (e.g., cranial penetration). In certain embodiments, distance D₁ is about 75% of the distance D₂. In a particular embodiment, distance D₁ is about 30 mm and distance D₂ is about 40 mm. In other embodiments, D₁ may be approximately 1-3 centimeters (cm) and D₂ may be approximately 3-4 cm. Each sensing unit of the sensor 12, for example the first and second sensing units 80, 82 of FIG. 3, may have similar emitter-detector spacing. For example, the third distance D₃ in FIG. 3 represents a shallower optical path and the emitter-detector spacing at the fourth distance D₄, greater than the third distance D₃, represents a deeper optical path for tissue penetration. In certain embodiments, distance D₃ is about 75% of the distance D₄. In a particular embodiment, distance D₃ is about 30 mm and distance D₄ is about 40 mm. In other embodiments, D₃ may be approximately 1-3 centimeters (cm) and D₄ may be approximately 3-4 cm.

Turning again to FIG. 3, the first and the second sensing units 80, 82 may be arranged in a generally X-shaped configuration such that a first axis 84 of the first sensing unit 80 intersects a second axis 86 of the second sensing unit 82. Where the first and the second sensing units 80, 82 both include the first detector 18A (e.g., light from the first emitter 16A and light from the second emitter 16B is detected at the first detector 18A and is utilized to determine the first and the second regional oxygen saturation measurements), a point of intersection 88 of the first axis 84 and the second axis 86 is located at the first detector 18A, as shown. In some such embodiments, the first axis 84 and the second axis 86 are positioned at an angle 90 relative to one another. The angle 90 may be any suitable angle. For example, the angle may be between about 10-90 degrees, 20-80 degrees, 30-70 degrees, 40-60 degrees, or between about 45-55 degrees.

As shown in FIG. 3, the one or more emitters 16 and the one or more detectors 18 may be arranged to provide (e.g., form) a two-dimensional monitoring area 104 (e.g., a two-dimensional area of contact between the sensing units and a surface of the patient's skin when applied to the patient). In the embodiment of FIG. 3, the two-dimensional monitoring area 104 is defined by the first emitter 16A, the second emitter 16B, the second detector 18B, and the third detector 18C. In the illustrated embodiment, the angle 90 and/or the distances d between the one or more emitters 16 and the one or more detectors 18 may affect the size and geometry (e.g., shape) of the two-dimensional monitoring area 104. For example, the first and second sensing units 80, 82, and thus, the two-dimensional monitoring area 104 may extend about 0.5-6 cm along a vertical axis 100 of the sensor 12 and/or about 3-8 cm along a horizontal axis 102 of the sensor 12. The two-dimensional monitoring area 104 may have any suitable size. In certain embodiments, the two-dimensional monitoring area 104 may be approximately 6, 7, or 8 cm². In some embodiments, the two-dimensional monitoring area 104 may be greater than approximately 5, 10, 15, 20, or 25 cm². In some embodiments, the two-dimensional monitoring area 104 may be about 5-20, 6-15, or 7-10 cm². A larger two-dimensional monitoring area 104 may result in a larger volume of tissue being monitored, which results in decreased sensitivity to variations in sensor placement. For example, in medical settings, sensors 12 are often applied to the patient at a first (e.g., original) location, removed from the patient, and subsequently replaced on the patient close to or in the same general region of the original location (e.g., the forehead, the abdomen, or the like). Sensors 12 configured to monitor a larger volume of tissue may result in a larger fraction of the volume of tissue that is monitored before replacement being monitored after replacement, resulting in decreased variation in signals before and after replacement of the sensor 12. Moreover, sensors 12 configured to monitor a larger volume of tissue may result in decreased sensitivity to localized physiological variations. This decreased sensitivity is desirable since clinicians may be interested in monitoring a whole organ (e.g., the brain) rather than a small fraction of the organ.

Further, the first detector 18A may be smaller in size than the second and/or third detector 18B, 18C in order to equalize the differences in light intensity received/detected due to the distance of the detector from corresponding emitters 16A, 16B. Thus, the size of the detector 18A, 18B, 18C may be a function of the distance of the detector 18A, 18B, 18C from the corresponding emitter 16A, 16B. The size and/or the distance of the detectors 18A, 18B, 18C from the corresponding emitter 16A, 16B may be a function of a desired mean path length of light traversing through human tissue. As noted above, although two emitters 16A, 16B and three detectors 18A, 18B, 18C are depicted in FIG. 3, any suitable number of detectors 18 or emitters 16 may be utilized. Further, although the two sensing units 80, 82 each include two detectors 18, it should be understood that three or more detectors 18 may be provided in the set of detectors 18 for one or more of the sensing units 80, 82. Additionally, although two sensing units 80, 82 for obtaining two regional saturation measurements are depicted, any suitable number of sensing units may be provided.

With reference to FIG. 3, the monitor 14 may be configured to receive signals from each of the detectors 18 of each sensing unit 80, 82 and to process the signals to determine the first and second regional oxygen saturation measurements. In certain embodiments, the monitor 14 may be configured to analyze the signals received and/or to analyze the first and second regional oxygen saturation measurements (e.g., quantitative values) to make various determinations. For example, the monitor 14 may compare the signals and/or the measurements. The monitor 14 may determine an average of the measurements. The monitor 14 may determine a median of the measurements, which may facilitate exclusion of outliers, for example. The monitor 14 may omit one or more measurements that exceed a predetermined threshold or are greater than a certain number of standard deviations away from the predetermined threshold or the average, which may facilitate exclusion of outliers, for example. The monitor 14 may determine a signal-to-noise ratio (SNR) of each of the signals, arbitrate based on the SNR, calculate a weighted average based on the SNR (e.g., weights are determined based on the SNR and the weighted measurements are averaged), and/or omit one or more of the signals having an SNR below a predetermined SNR threshold. In some embodiments, the monitor 14 may determine a quality metric (e.g., a confidence metric) based on variations (e.g., differences or degree of correlation) between the signals and/or the measurements. In certain embodiments, the monitor 14 may determine and/or provide the quality metric of each signal, each measurement, or of the average or the mean calculated by the monitor 14, for example. In some embodiments, the monitor 14 may evaluate which signal(s) or measurement(s) should be used to determine the patient's regional oxygen saturation based on the emitter-detector spacing, the signal-to-noise ratio, and/or the quality metric.

In certain embodiments, the monitor 14 may be configured to compare the signals and/or the measurements to identify and/or to evaluate variations in the patient's tissue. In particular, the monitor 14 may evaluate variations in the patient's tissue underlying the sensor 12 and/or the two-dimensional monitoring area 104, for example. It is generally desirable to place regional oxygen saturation sensors over a uniform and/or structurally consistent tissue bed to generate accurate, reliable, and/or repeatable regional oxygen saturation measurements. Anatomical differences in tissue, fluid layers, and/or bone structures underlying the sensor 12 and/or the two-dimensional monitoring area 104 may affect a path traveled by the emitted light and, thus, the regional oxygen saturation measurements. Accordingly, it may be desirable for the sensor 12 to compare the signals generated by each detector and/or the regional saturation measurements determined based on the signals to evaluate the tissue bed and/or to determine whether the sensor 12 should be repositioned. For example, if the regional oxygen saturation measurements obtained by the different sensing units (e.g., the first and the second sensing units 80, 82) vary by more than a predetermined variation threshold (e.g., by more than 1%, 3%, 5%, 10% or more), the monitor 14 may determine that the sensor 12 is not positioned over a suitably uniform tissue bed. In such circumstances, the monitor 14 may provide an indication that the sensor 12 should be repositioned. The indication provided by the monitor 14 may take any suitable form, including an audible alarm or a visual alarm or message on the display 20 of the monitor 14. As discussed in more detail below, in embodiments where the sensor 12 includes more than two sensing units for obtaining more than two regional saturation measurements, the monitor 14 may be configured to provide an indication of which portion of the sensor 12 should be repositioned. For example, if all signals or measurements are within the predetermined variation threshold except for one sensing unit having one or more detectors 18 located near a top edge 108 (e.g., a first edge) of the sensor 12, the monitor 14 may be configured to indicate that the sensor 12 should be moved lower (e.g., in a first direction), as shown by arrow 110, on the patient's tissue.

In some embodiments, the first and the second regional oxygen saturation measurements may be separately displayed on the monitor display 20 as a numeric or other such quantified value, constituting an approximately instantaneous real-time value, and/or as a point in a graphical plot, representing a succession of such values taken over time. In certain embodiments, a single calculated regional oxygen saturation value may be displayed on the monitor display 20, and the single calculated value may be based on any of the above processing techniques and thus may be an average, a median, or the like. In some embodiments, an indication of the quality metric may be provided on the monitor display 20, such as via a numerical value or other suitable indicator. In some embodiments, the monitor display 20 may be configured to provide an indication of which of the sensing units were utilized to determine the displayed regional oxygen saturation value. For example, the monitor display 20 may indicate that only the first sensing unit 80 was used, that only the second sensing unit 82 was used, or that an average of the first and the second regional saturation measurements obtained by both sensing units 80, 82 was calculated and displayed. In some embodiments, the monitor display 20 may provide an indication related to a reason that certain signals or measurements are not provided, such as due to a variance between the measurements being above the predetermined variance threshold, for example.

Although described with respect to the embodiment of FIG. 3, it should be understood that such processing techniques may be adapted for use with any of the embodiments of the sensor 12 disclosed herein. For example, where the sensor 12 includes more than two sensing units for obtaining regional saturation measurements, the monitor 14 may be configured to combine all of the measurements (e.g., average or median), omit certain measurements based on SNR, or the like. Additionally, the monitor display 20 may be adapted to provide the appropriate information for the particular sensor configuration, including in any of the manners discussed above with respect to FIG. 3.

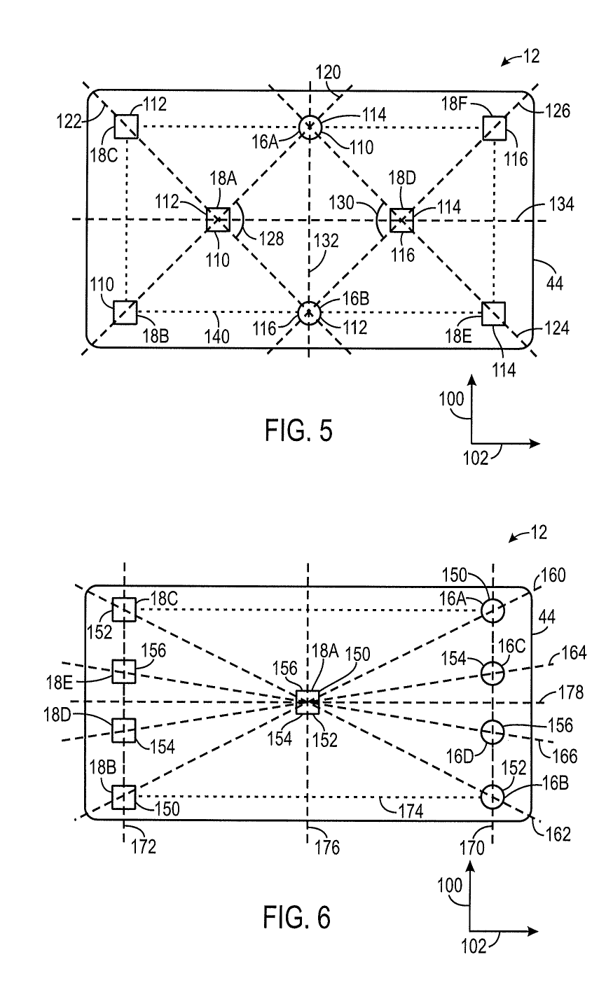

With the foregoing in mind, additional embodiments of the sensor 12 are provided in FIGS. 5-10. FIG. 5 illustrates an embodiment of the sensor 12 having four sensing units 110, 112, 114, 116 for obtaining four regional saturation measurements. As shown, the sensor 12 includes two emitters 16A, 16B and six detectors 18A, 18B, 18C, 18D, 18E, 18F. As disclosed above with respect to FIG. 3, the first emitter 16A is configured to be used with the first detector 18A and the second detector 18B to obtain a first regional oxygen saturation measurement when the sensor 12 is applied to the patient. Thus, the first detector 18A and the second detector 18B form a first set of detectors, and the first emitter 16A and the first set of detectors (i.e., the first detector 18A and the second detector 18B) form a first sensing unit 110 for obtaining the first regional saturation measurement. As depicted, the sensor 12 also includes the second emitter 16B configured to be used with the first detector 18A and the third detector 18C to obtain a second regional oxygen saturation measurement when the sensor 12 is applied to the patient. Thus, the first detector 18A and the third detector 18C form a second set of detectors, and the second emitter 16B and the second pair of detectors (i.e., the first detector 18A and the third detector 18C) form a second sensing unit 112 for obtaining the second regional oxygen saturation measurement.

As shown, the sensor 12 of FIG. 5 includes two additional sensing units 114, 116. Thus, the first emitter 16A is configured to be used with the fourth detector 18D and the fifth detector 18E to obtain a first regional oxygen saturation measurement when the sensor 12 is applied to the patient. Thus, the fourth detector 18D and the fifth detector 18E form a third set of detectors, and the first emitter 16A and the third pair of detectors (i.e., the fourth detector 18D and the fifth detector 18E) form a third sensing unit 114 for obtaining the third regional saturation measurement. As depicted, the sensor 12 also includes the second emitter 16B configured to be used with the fourth detector 18D and the sixth detector 18F to obtain a fourth regional oxygen saturation measurement when the sensor 12 is applied to the patient. Thus, the fourth detector 18D and the sixth detector 18F form a fourth set of detectors, and the second emitter 16B and the fourth pair of detectors (i.e., the fourth detector 18D and the sixth detector 18F) form a fourth sensing unit 116 for obtaining the fourth regional oxygen saturation measurement.

In the illustrated embodiment, the first and third sensing units 110, 114 include the first emitter 16A, and the second and the fourth sensing units 112, 116 include the second emitter 16B. Additionally, the first and second sensing units 110, 112 include the first detector 18A, and the third and fourth sensing units 114, 116 include the fourth detector 18D. The emitter 16 and corresponding detectors 18 of each sensing unit 110, 112, 114, 116 may be positioned along a respective axis of the sensing unit 110, 112, 114, 116, in some embodiments. For example, the first emitter 16A and the first pair of detectors 18A, 18B may be positioned along a first axis 120. The second emitter 16B and the second pair of detectors 18A, 18C may be positioned along a second axis 122. The first emitter 16A and the third pair of detectors 18D, 18E may be positioned along a third axis 124. The second emitter 16B and the fourth pair of detectors 18D, 18F may be positioned along a fourth axis 126.

The distance d between the emitter 16 and corresponding detectors 18 of each sensing unit 110, 112, 114, 116 may be any suitable distance, including those discussed above with respect to FIG. 3. Similarly, a first angle 128 between the first axis 120 and the second axis 122 and/or a second angle 130 between the third axis 124 and the fourth axis 126 may be any suitable angle, including those discussed above with respect to FIG. 3. In certain embodiments, the first angle 128 and the second angle 130 may be equal, although in other embodiments the first angle 128 and the second angle 130 may differ to facilitate monitoring various different tissues and to facilitate application of the sensor 12 to various different tissue sites. As shown, the emitters 16 and the detectors 18 are arranged symmetrically (e.g., a mirror-image) about a vertical axis of symmetry 132 and/or a horizontal axis of symmetry 134, which may also be vertical and horizontal center lines of the sensor 12. The emitters 16 are positioned along the vertical axis of symmetry 132 and/or the first and the fourth detectors 18A, 18D are positioned along the horizontal axis of symmetry 134.

The one or more emitters 16 and the one or more detectors 18 of the sensor 12 of FIG. 5 may be arranged to provide (e.g., form) a two-dimensional monitoring area 140. In the embodiment shown in FIG. 5, the two-dimensional monitoring area 140 is defined by the second detector 18B, the third detector 18C, the fifth detector 18E, and the sixth detector 18F, although the first emitter 16A and/or the second emitter 16B may be positioned to define the two-dimensional monitoring area 104 in certain embodiments. The angles 128, 130 and/or the distances d between the one or more emitters 16 and the one or more detectors 18 may affect the size and geometry (e.g., shape) of the two-dimensional monitoring area 140. For example, some or all of the sensing units 110, 112, 114, 116, and thus the two-dimensional monitoring area 140, may extend about 0.5-6 cm along the vertical axis 100 of the sensor 12 and/or about 3-8 cm along the horizontal axis 102 of the sensor 12. As discussed above with respect to FIG. 3, the two-dimensional monitoring area 140 may have any suitable size. In certain embodiments, the two-dimensional monitoring area 140 may be approximately 6, 7, or 8 cm². In some embodiments, the two-dimensional monitoring area 140 may be greater than approximately 5, 10, 15, 20, or 25 cm². In some embodiments, the two-dimensional monitoring area 104 may be about 5-20, 6-15, or 7-10 cm².

The signals received from the detectors 18 of the sensor 12 of FIG. 5 and/or the regional oxygen saturation measurements determined based on the received signals, may be processed and/or displayed in a similar manner as disclosed above with respect to FIG. 3. For example, the four regional saturation measurements determined based on the signals received from the four sensing units 110, 112, 114, 116 may be compared, averaged, and/or displayed. The monitor 14 may be configured to evaluate uniformity of the tissue underlying the sensor 12 based on a variance between the signals and/or measurements, for example. In some embodiments, the monitor 14 may be configured to determine whether the sensor 12 should be repositioned based on the variance, as set forth above. As compared with the sensor 12 of FIG. 3, the configuration of the sensor 12 in FIG. 5 may generally monitor a larger area (e.g., may have a larger two-dimensional monitoring area 140) and/or may provide more signals and measurements that can be utilized in the above-described processing steps, for example.

FIG. 6 illustrates an embodiment of the sensor 12 having four sets of optical components for obtaining four regional saturation measurements. As shown, the sensor 12 includes four emitters 16A, 16B, 16C, 16D and five detectors 18A, 18B, 18C, 18D, 18E arranged into four sensing units 150, 152, 154, 156. As in FIG. 3, the first emitter 16A is configured to be used with the first detector 18A and the second detector 18B to obtain a first regional oxygen saturation measurement when the sensor 12 is applied to the patient. Thus, the first detector 18A and the second detector 18B form a first set of detectors, and the first emitter 16A and the first set of detectors (i.e., the first detector 18A and the second detector 18B) form a first sensing unit 150 for obtaining the first regional saturation measurement. As depicted, the sensor 12 also includes the second emitter 16B configured to be used with the first detector 18A and the third detector 18C to obtain a second regional oxygen saturation measurement when the sensor 12 is applied to the patient. Thus, the first detector 18A and the third detector 18C form a second set of detectors, and the second emitter 16B and the second set of detectors (i.e., the first detector 18A and the third detector 18C) form a second sensing unit 152 for obtaining the second regional oxygen saturation measurement.

As shown, the sensor 12 of FIG. 6 includes two additional sensing units 154, 156. The third emitter 16C is configured to be used with the first detector 18A and the fourth detector 18D to obtain a first regional oxygen saturation measurement when the sensor 12 is applied to the patient. Thus, the first detector 18A and the fourth detector 18D form a third set of detectors, and the third emitter 16C and the third set of detectors (i.e., the first detector 18A and the fourth detector 18D) form a third sensing unit 154 for obtaining the third regional saturation measurement. As depicted, the sensor 12 also includes the fourth emitter 16D configured to be used with the first detector 18A and the fifth detector 18E to obtain a fourth regional oxygen saturation measurement when the sensor 12 is applied to the patient. Thus, the first detector 18A and the fifth detector 18E form a fourth set of detectors, and the fourth emitter 16D and the fourth set of detectors (i.e., the first detector 18A and the fifth detector 18E) form a fourth sensing unit 156 for obtaining the fourth regional oxygen saturation measurement.

In the illustrated embodiment, the first sensing unit 150 is positioned along a first axis 160, the second sensing unit 152 is positioned along a second axis 162, the third sensing unit 154 is positioned along a third axis 164, and the fourth sensing unit 156 is positioned along the fourth axis 166. Each of the sensing units 150, 152, 154, 156 includes the first detector 18A, and the axes 160, 162, 164, 166 intersect at the first detector 18A. As shown, the emitters 16A, 16B, 16C, 16D are arranged linearly along line 170 and at least some of the detectors 18B, 18C, 18D, 18E are arranged linearly along line 172. In certain embodiments, all of the detectors 18 positioned relatively far from the corresponding emitter 16 of respective sensing units may be arranged linearly, such as along line 172, as shown in FIG. 6. As shown, a two-dimensional monitoring area 174 is defined by the emitters 16A, 16B, 16C, 16D and the detectors 18B, 18C, 18D, 18E, that are positioned far from the corresponding emitter 16.

The distance between adjacent emitters 16 along line 170 may be uniform or may vary, and similarly, the distance between adjacent detectors 18 along line 172 may be uniform or may vary. Additionally, the distance between the emitter 16 and the corresponding detectors 18 of each sensing unit 150, 152, 154, 156 may be uniform across sensing units or may vary. As shown, the far detectors 18B, 18C, 18D, 18E are positioned on a first side of a vertical axis 176 of the sensor 12, which may be a vertical center axis of the sensor 12. In some such embodiments, the emitters 16A, 16B, 16C, 16D are positioned on a second side of the vertical axis 170 of the sensor 12. In certain embodiments, the emitters 16 and the detectors 18 may be arranged symmetrically about a horizontal axis 178 of the sensor 12, which may be a horizontal center axis of the sensor 12. The first detector 18A may be located in any suitable position between the emitters 16 and the far detectors 18B, 18C, 18D, 18E.

The signals received from the detectors 18 of the sensor 12 of FIG. 6 and/or the regional oxygen saturation measurements determined based on the received signals, may be processed and/or displayed in a similar manner as disclosed above with respect to FIG. 3. For example, the four regional saturation measurements determined based on the signals received from the four sensing units 150, 152, 154, 156 may be compared, averaged, and/or displayed. The monitor 14 may be configured to evaluate uniformity of the tissue underlying the sensor 12 based on a variance between the signals and/or measurements, for example. In some embodiments, the monitor 14 may be configured to determine whether the sensor 12 should be repositioned based on the variance, as set forth above. As compared with the sensor 12 of FIG. 3, the configuration of the sensor 12 in FIG. 5 may generally monitor a larger area (e.g., may have a larger two-dimensional monitoring area 140) and/or may provide more signals and measurements that can be utilized in the above-described processing steps, for example.

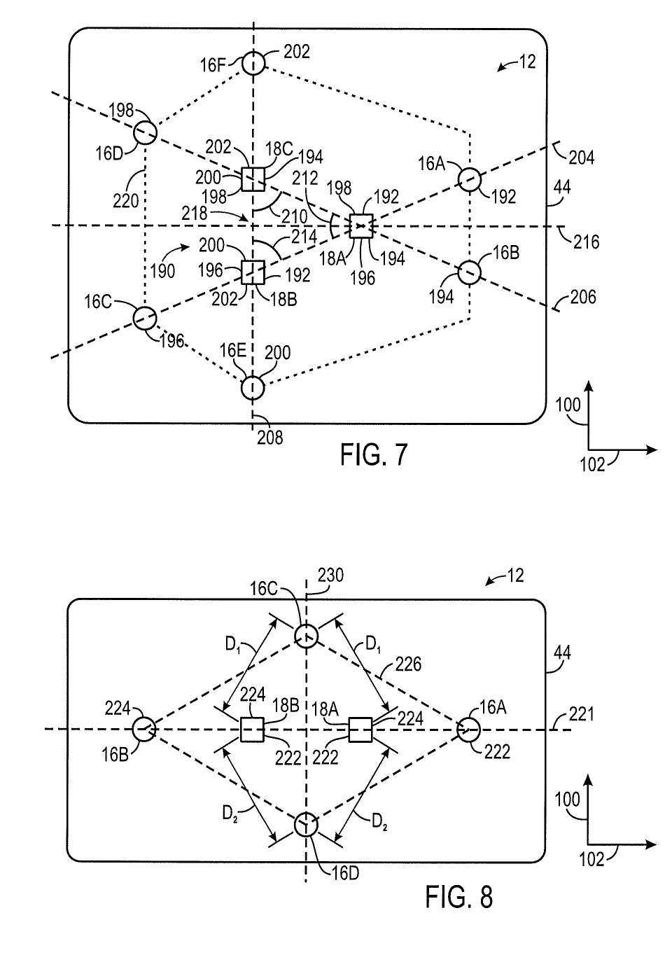

FIG. 7 illustrates an embodiment of the sensor 12 having six sensing units for obtaining six regional saturation measurements. As shown, the sensor 12 includes six emitters 16A, 16B, 16C, 16D, 16E, 16F and three detectors 18A, 18B, 18C. The emitters 16 are generally arranged about the detectors 18, which are positioned on an interior portion 190 of the sensor 12 relative to the emitters 16. Positioning the detectors 18 in the interior portion 190 may facilitate light shielding or blocking of ambient light from the detectors 18, for example. As disclosed above with respect to FIG. 3, the first emitter 16A is configured to be used with the first detector 18A and the second detector 18B to obtain a first regional oxygen saturation measurement when the sensor 12 is applied to the patient. Thus, the first detector 18A and the second detector 18B form a first set of detectors, and the first emitter 16A and the first set of detectors (i.e., the first detector 18A and the second detector 18B) form a first sensing unit 192 for obtaining the first regional saturation measurement. As depicted, the sensor 12 also includes the second emitter 16B configured to be used with the first detector 18A and the third detector 18C to obtain a second regional oxygen saturation measurement when the sensor 12 is applied to the patient. Thus, the first detector 18A and the third detector 18C form a second set of detectors, and the second emitter 16B and the second set of detectors (i.e., the first detector 18A and the third detector 18C) form a second sensing unit 194 for obtaining the second regional oxygen saturation measurement.

As shown, the sensor 12 of FIG. 7 includes additional sensing units 196, 198, 200, 202. The third emitter 16C is configured to be used with the first detector 18A and the second detector 18B to obtain a third regional oxygen saturation measurement when the sensor 12 is applied to the patient. Thus, like the first emitter 16A, the third emitter 16C is also configured to be used with the first set of detectors (e.g., the first detector 18A and the second detector 18B) to obtain a regional saturation measurement. However, the third emitter 16C is positioned proximate to the second detector 18B and distal from the first detector 18A, while the first emitter 16A is positioned proximate to the first detector 18A and distal from the second detector 18B. The third emitter 16C and the first pair of detectors form a third sensing unit 196 for obtaining the third regional saturation measurement.

As depicted, the sensor 12 also includes the fourth emitter 16D configured to be used with the third detector 18C and the first detector 18A to obtain a fourth regional oxygen saturation measurement when the sensor 12 is applied to the patient. Thus, like the second emitter 16B, the fourth emitter 16D is also configured to be used with the second set of detectors (e.g., the first detector 18A and the third detector 18C) to obtain a regional saturation measurement. However, the fourth emitter 16D is positioned proximate to the third detector 18C and distal from the first detector 18A, while the second emitter 16B is positioned proximate to the first detector 18A and distal from the third detector 18C. The fourth emitter 16D and the second set of detectors form a fourth sensing unit 198 for obtaining the fourth regional saturation measurement.

In certain embodiments, a fifth emitter 16E may be provided and may be configured to be used with the second detector 18B and the third detector 18C to obtain a fifth regional oxygen saturation measurement when the sensor 12 is applied to the patient. Thus, the second detector 18B and the third detector 18C form a third set of detectors, and the fifth emitter 16E and the third set of detectors (i.e., the second detector 18B and the third detector 18C) form a fifth sensing unit 200 for obtaining the fifth regional oxygen saturation measurement. A sixth emitter 16F may be provided and may be configured to be used with the second detector 18B and the third detector 18C to obtain a sixth regional saturation measurement when the sensor 12 is applied to the patient. Thus, like the fifth emitter 16E, the sixth emitter 16F is also configured to be used with the third set of detectors (e.g., the second detector 18B and the third detector 18C) to obtain a regional saturation measurement. However, the sixth emitter 16F is positioned proximate to the third detector 18C and distal from the second detector 18B, while the fifth emitter 16E is positioned proximate to the second detector 18B and distal from the third detector 18C. The sixth emitter 16F and the third set of detectors form a sixth sensing unit 202 for obtaining the sixth regional saturation measurement. As shown, in certain embodiments, the first and/or third sensing units 192, 196 may be positioned along a first axis 204, the second and/or fourth sensing units 194, 198 may be positioned along a second axis 206, and/or the fifth and/or sixth sensing units 200, 202 may be positioned along a third axis 208.

In the illustrated embodiment having three detectors 18A, 18B, 18C, the detectors 18A, 18B, 18C, and/or the axes 204, 206, 208 may be arranged in a generally triangular orientation. Angles 210, 212, 214 defined by the axes 204, 206, 208 extending between the detectors 18A, 18B, 18C may be any suitable angle, such as approximately 10-170 degrees, 15-110 degrees, 20-90 degrees, 25-80 degrees, 30-70 degrees, or 35-60 degrees, for example. Additionally, some or all of the angles 210, 212, 214 may be equal to one another or may vary. In certain embodiments, the emitters 16 and the detectors 18 may be symmetrically arranged about an axis of symmetry 216, which may also be a horizontal axis, a vertical axis, a center horizontal axis, or a center vertical axis of the sensor, depending on the arrangement of the emitters 16 and detectors 18 relative to the sensor body 44, for example. As shown, the axis of symmetry 216 may extend through the first detector 18A and/or may intersect the axis 208 at a midpoint 218 centered between the second detector 18B and the third detector 18C and/or centered between the fifth emitter 16E and the sixth emitter 16F, as shown. In the embodiment of FIG. 7, one or more of the emitters 16 positioned about the detectors 18 may define a two-dimensional monitoring area 220.

It should be understood that the emitters 16 and the detectors 18 of FIG. 7 may be arranged in any of a variety of configurations. For example, the detectors 18A, 18B, 18C may be equidistant from one another and may form an equilateral triangle (e.g., angles 210, 212, 214 may be equal to one another) having a center point (not shown) at a center of the equilateral triangle. In such cases, the emitters 16A, 16B, 16C, 16D, 16E, 16F may be arranged about the detectors 18A, 18B, 18C and may be equidistant from the center point, and thus, the sensor 12 may have a tri-fold symmetry about the center point positioned within the equilateral triangle formed by the detectors 18A, 18B, 18C. Such a configuration may be particularly useful for determining a proper location for the sensor 12. As noted above, by way of example, differences in signal quality between the six sensing units 192, 194, 196, 198, 200, 202 may be used to indicate to the user a direction in which to move the sensor 12 to increase the symmetry of signals obtained via the six sensing units 192, 194, 196, 198, 200, 202, and thus, better monitor a particular organ (e.g., a kidney).

The signals received from the detectors 18 of the sensor 12 of FIG. 7 and/or the regional oxygen saturation measurements determined based on the received signals, may be processed and/or displayed in a similar manner as disclosed above with respect to FIG. 3. For example, the six regional saturation measurements determined based on the signals received from the six sensing units 192, 194, 196, 198, 200, 202 may be compared, averaged, and/or displayed. The monitor 14 may be configured to evaluate uniformity of the tissue underlying the sensor 12 based on a variance between the signals and/or measurements, for example. In some embodiments, the monitor 14 may be configured to determine whether the sensor 12 should be repositioned based on the variance, as set forth above. As compared with the sensor 12 of FIG. 3, the configuration of the sensor 12 in FIG. 7 may generally monitor a larger area (e.g., may have a larger two-dimensional monitoring area 220) and/or may provide more signals and measurements that can be utilized in the above-described processing steps, for example.

FIG. 8 illustrates an embodiment of the sensor 12 having two sensing units for obtaining two regional saturation measurements. As shown, the sensor 12 includes a first emitter 16A, a second emitter 16B, a first detector 18A, and a second detector 18B, each positioned linearly along a first axis 221. In the depicted embodiment, the first emitter 16A is configured to be used with the first detector 18A and the second detector 18B to obtain a first regional oxygen saturation measurement when the sensor 12 is applied to the patient. Thus, the first detector 18A and the second detector 18B form a first set of detectors, and the first emitter 16A and the first set of detectors (i.e., the first detector 18A and the second detector 18B) form a first sensing unit 222 for obtaining the first regional saturation measurement. As depicted, the sensor 12 also includes the second emitter 16B that is also configured to be used with the first detector 18A and the second detector 18B to obtain a second regional oxygen saturation measurement when the sensor 12 is applied to the patient. Thus, like the first emitter 16A, the second emitter 16B is also configured to be used with the first set of detectors (e.g., the first detector 18A and the second detector 18B) to obtain a regional saturation measurement. However, the second emitter 16B is positioned proximate to the second detector 18B and distal from the first detector 18A, while the first emitter 16A is positioned proximate to the first detector 18A and distal from the second detector 18B. The second emitter 16B and the first set of detectors form a second sensing unit 224 for obtaining the second regional saturation measurement.

In certain embodiments, the sensor 12 may include one or more additional emitters 16 that may be used to obtain additional data, such as information related to the uniformity of the tissue bed underlying the sensor 12 and/or a two-dimensional monitoring area 226. The one or more additional emitters 16 may emit one or more wavelengths of light, and in some embodiments, may emit only a single wavelength that enables identification of tissue or structural properties. By way of example, a third emitter 16C and/or a fourth emitter 16D may be provided in the sensor 12 of FIG. 8. The one or more additional emitters 16 may be positioned in any suitable location, although as shown the third emitter 16C is positioned on one side of the axis 221, and the fourth emitter 16D is positioned on another side of the first axis 221. Additionally, in the illustrated embodiment, the third emitter 16C is positioned a first distance D₁ relative to each of the detectors 18A, 18B, while the fourth emitter 16D is positioned a second distance D₂ relative to each of the detectors 18A, 18B. In some embodiments, some or all of the emitters 16 for detecting tissue uniformity may be equal distances from each of the detectors (e.g., the first distance D₁ may be equal to the second distance D₂). As shown, the third emitter 16C and the fourth emitter 16D are positioned along a second axis 230.

The light emitted by the third emitter 16C may be received at the first detector 18A and/or the second detector 18B. The signals generated by the detectors 18A, 18B may not be utilized for regional saturation measurements as the light received at each of the detectors 18A, 18B from the third emitter 16C traveled the same first distance D₁ and thus through the same tissue depth. However, the light received at each detector 18 may be utilized to determine tissue uniformity. For example, because the light emitted by the third detector 16A travels the first distance D₁ to reach the first and second detectors 18A, 18B, variations in the detected light at each detector 18A, 18B may indicate non-uniformity of the tissue between the third emitter 16C and the detectors 18A, 18B. Where D₁ is equal to D₂, variations in the detected light emitted by each emitter 16A, 16B and detected at each detector 18A, 18B may indicate non-uniformity of the tissue between the emitters 16A, 16B and the detectors 18A, 18B about the first axis 221, for example.

The signals received from the detectors 18 of the sensor 12 of FIG. 8 and/or the regional oxygen saturation measurements determined based on the received signals, may be processed and/or displayed in a similar manner as disclosed above with respect to FIG. 3. For example, the two regional saturation measurements determined based on the signals received from the two sensing units 222, 224 may be compared, averaged, and/or displayed. The monitor 14 may be configured to evaluate uniformity of the tissue underlying the sensor 12 based on a variance between the signals and/or measurements, for example. As discussed above, additional emitters 16 may be used to determine additional information related to uniformity of the tissue. In some embodiments, the monitor 14 may be configured to determine whether the sensor 12 should be repositioned based on the variance, as set forth above. As compared with the sensor 12 of FIG. 3, the configuration of the sensor 12 in FIG. 8 provides a relatively compact, linear sensor that may be desirable for certain tissue sites, while still obtaining multiple regional saturation measurements and/or monitoring tissue uniformity, for example.

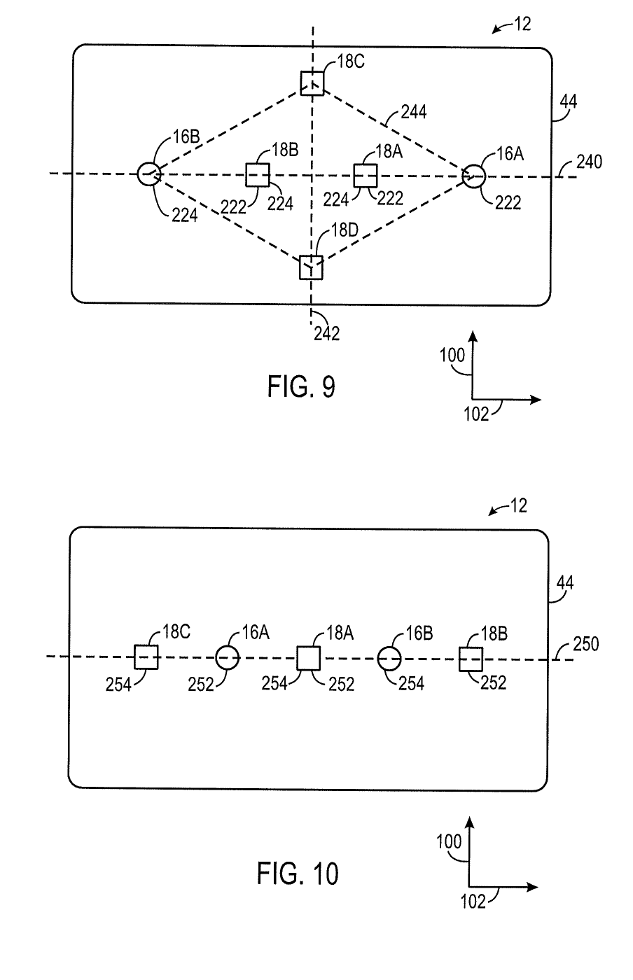

FIG. 9 illustrates an embodiment of the sensor 12 having two sensing units for obtaining two regional saturation measurements. As shown, the sensor 12 includes a first emitter 16A, a second emitter 16B, a first detector 18A, and a second detector 18B positioned linearly along the first axis 240 in a similar configuration as discussed above with respect to FIG. 8. Additionally, a third detector 18C and/or a fourth detector 18D may be provided, such as along a second axis 242, as shown. The third detector 18C and/or the fourth detector 18D may be configured to receive light from one or both of the emitters 16A, 16B, and the signals generated by the detectors 18C, 18D based on the light received may be utilized to calculate regional saturation measurements and/or to evaluate the uniformity of the tissue underlying the sensor 12, as discussed above.

In certain embodiments, the first emitter 16A and the second emitter 16B may be configured to emit alternating wavelengths of light. For example, the first emitter 16A may emit a first wavelength λ₁ and a third wavelength λ₃, while the second emitter 16B may emit a second wavelength λ₂ and the fourth wavelength λ₄. In some embodiments, the first wavelength λ₁ is less than the second wavelength λ₂, which is less than the third wavelength λ₃, which is less than the fourth wavelength λ₄. For example, the first emitter 16A may emit the first wavelength λ₁ of about 724 nm and the third wavelength λ₃ of about 812 nm, while the second emitter 16B may emit the second wavelength λ₂ of about 770 nm and the fourth wavelength λ₄ of about 850 nm, although any suitable wavelengths may be emitted by the emitters 16A, 16B. Multiple pairs of different wavelengths may be suitable for obtaining various physiological parameters and for determining regional oxygen saturation. Thus, using a first pair of suitable wavelengths, the first wavelength λ₁ and the third wavelength λ₃, to calculate a first regional oxygen saturation measurement using the first sensing unit 222 and using a second, different pair of suitable wavelengths, the second wavelength λ₂ and the fourth wavelength λ₄, to calculate a second regional saturation measurement using the second sensing unit 224 may provide redundancy and robustness in monitoring, along with reduced part count and associated costs as compared to the system having each emitter 16 configured to emit all of the wavelengths (e.g., the first wavelength λ₁, the second wavelength λ₂, the third wavelength λ₃, and the fourth wavelength λ₄). Although emitting different wavelengths of light from the respective emitters 16 of different sensing units to calculate regional oxygen saturation are discussed herein in the context of the sensor 12 of FIG. 9, it should be understood that any of the embodiments of the sensor 12 disclosed herein may utilize different (e.g., alternating) wavelengths.

The signals received from the detectors 18 of the sensor 12 of FIG. 9 and/or the regional oxygen saturation measurements determined based on the received signals, may be processed and/or displayed in a similar manner as disclosed above with respect to FIG. 3. For example, the two regional saturation measurements determined based on the signals received from the two sensing units 222, 224 may be compared, averaged, and/or displayed. The monitor 14 may be configured to evaluate uniformity of the tissue underlying the sensor 12 based on a variance between the signals and/or measurements, for example. In some embodiments, the monitor 14 may be configured to determine whether the sensor 12 should be repositioned based on the variance, as set forth above. As compared with the sensor 12 of FIG. 3, the configuration of the sensor 12 in FIG. 9 provides a relatively compact, linear sensor that may be desirable for certain tissue sites, while still obtaining multiple regional saturation measurements and/or monitoring tissue uniformity, for example. Where additional detectors 18C, 18D are provided, regional saturation measurements may be based on light received at the additional detectors 18C, 18D, and thus, the regional saturation measurements may be based on a relatively larger tissue area (e.g., a two-dimensional monitoring area 244). Furthermore, in any of the embodiments disclosed herein, given the reciprocal nature of light and most electromagnetic waves travelling through tissue, positions of emitters 16 and detectors 18 in a given sensing unit may be exchanged with each other without affecting functionality. That is, one emitter 16 may be moved to a location of one detector 18 and vice-versa. In certain applications, it may be preferred to keep detectors 18 closer to the center of the sensor 12 since then the detectors 18 are better shielded from ambient light.

FIG. 10 illustrates a sensor 12 having two sensing units configured to obtain two regional saturation measurements. As shown, the emitters 16 and the detectors 18 are arranged linearly along an axis 250 in an alternating pattern, such that each emitter 16 is positioned between and/or is adjacent to two detectors 18. In such a configuration, the light emitted by the first emitter 16A and detected by the second detector 18B travels through the patient's tissue and under the second emitter 16B. Additionally, the light emitted by the second emitter 16B and detected by the third detector 18C travels through the patient's tissue and under the first emitter 16A.

In the depicted embodiment, the first emitter 16A is configured to be used with the first detector 18A and the second detector 18B to obtain a first regional oxygen saturation measurement when the sensor 12 is applied to the patient. Thus, the first detector 18A and the second detector 18B form a first set of detectors, and the first emitter 16A and the first set of detectors (i.e., the first detector 18A and the second detector 18B) form a first sensing unit 252 for obtaining the first regional saturation measurement. As depicted, the sensor 12 also includes the second emitter 16B that is also configured to be used with the first detector 18A and a third detector 18C to obtain a second regional oxygen saturation measurement when the sensor 12 is applied to the patient. Thus, the first detector 18A and the third detector 18C form a second set of detectors, and the second emitter 16B and the second set of detectors (e.g., the first detector 18A and the third detector 18C) form a second sensing unit 254 for obtaining the second regional saturation measurement.

The signals received from the detectors 18 of the sensor 12 of FIG. 10 and/or the regional oxygen saturation measurements determined based on the received signals, may be processed and/or displayed in a similar manner as disclosed above with respect to FIG. 3. For example, the two regional saturation measurements determined based on the signals received from the two sensing units 252, 254 may be compared, averaged, and/or displayed. The monitor 14 may be configured to evaluate uniformity of the tissue underlying the sensor 12 based on a variance between the signals and/or measurements, for example. As discussed above, additional emitters 16 may be used to determine additional information related to uniformity of the tissue. In some embodiments, the monitor 14 may be configured to determine whether the sensor 12 should be repositioned based on the variance, as set forth above. As compared with the sensor 12 of FIG. 3, the configuration of the sensor 12 in FIG. 10 provides a relatively compact, linear sensor that may be desirable for certain tissue sites, while still obtaining multiple regional saturation measurements and/or monitoring tissue uniformity, for example.

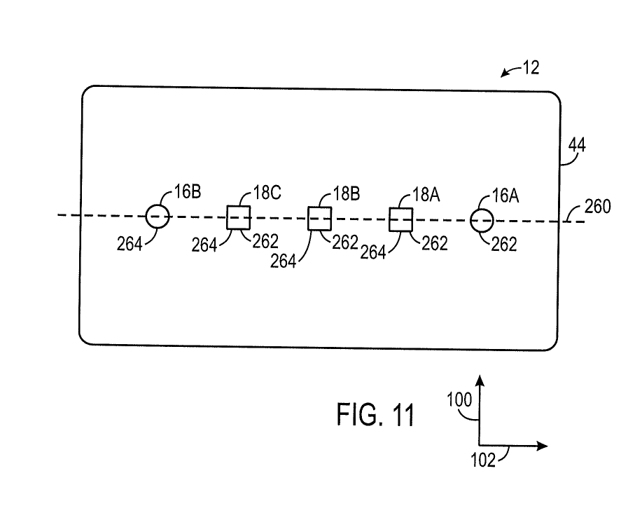

FIG. 11 illustrates a sensor 12 having at least one sensing unit, each sensing unit including one emitter 16 and three or more detectors 18 configured to receive light from the emitter 16 to generate a regional saturation measurement. As shown, a first emitter 16A, a first detector 18A, a second detector 18B, and a third detector 18C are arranged linearly along an axis 260 and may form a first sensing unit 262 configured to obtain a first regional saturation measurement. The light emitted by the first emitter 16A is detected at each of the three detectors 18A, 18B, 18C, and the signals generated by each of the three detectors 18A, 18B, 18C may be utilized by the monitor 14 to determine the first regional saturation measurement. In certain embodiments, additional emitters 16 and/or detectors 18 may be provided, such as along the axis 260. For example, as shown, a second emitter 16B is provided along the axis 260. The second emitter 16B and the three detectors 18A, 18B, 18C may form a second sensing unit 264 configured to obtain a second regional saturation measurement. Thus, light emitted by the second emitter 16B may be detected at each of the three detectors, 18A, 18B, 18C, and the signals generated by each of the three detectors 18A, 18B, 18C may be utilized by the monitor 14 to generate the second regional saturation measurement. As noted above, any of the embodiments disclosed herein may be adapted to include sensing units having three or more detectors 18.

The signals received from the detectors 18 of the sensor 12 of FIG. 11 and/or the regional oxygen saturation measurements determined based on the received signals, may be processed and/or displayed in a similar manner as disclosed above with respect to FIG. 3. For example, the two regional saturation measurements determined based on the signals received from the two sensing units 262, 264 may be compared, averaged, and/or displayed. The monitor 14 may be configured to evaluate uniformity of the tissue underlying the sensor 12 based on a variance between the signals and/or measurements, for example. As discussed above, additional emitters 16 may be used to determine additional information related to uniformity of the tissue. In some embodiments, the monitor 14 may be configured to determine whether the sensor 12 should be repositioned based on the variance, as set forth above. As compared with the sensor 12 of FIG. 3, the configuration of the sensor 12 in FIG. 11 provides a relatively compact, linear sensor that may be desirable for certain tissue sites, while still obtaining multiple regional saturation measurements and/or monitoring tissue uniformity, for example.

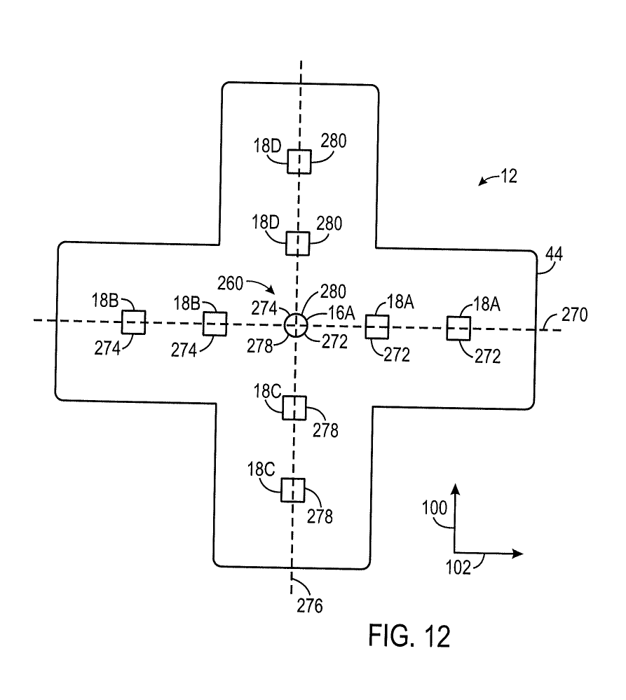

FIG. 12 illustrates a sensor 12 having four sensing units. As shown, the sensor 12 includes one emitter 16 and four sets of detectors 18A, 18B, 18C, 18D (e.g., each set includes multiple detectors 18) positioned about the emitter 16. The emitter 16 is positioned in a central portion 260 of the sensor 12, and each set of detectors 18A, 18B, 18C, 18D may extend radially-outwardly from the emitter 16, such as in a cross or X configuration, for example. As shown, the emitter 16 and a first set of detectors 18A may be positioned along a first axis 270 and may form a first sensing unit 272 for obtaining a first regional saturation measurement. The emitter 16 and a second set of detectors 18B may be positioned along the first axis 270 and may form a second sensing unit 274 for obtaining a first regional saturation measurement. As shown, the first set of detectors 18A may be positioned on one side of a second axis 276, while the second set of detectors 18B may be positioned on another side of the second axis 276. The emitter 16 and a third set of detectors 18C may be positioned along the second axis 276 and may form a third sensing unit 278 for obtaining a third regional saturation measurement. The emitter 16 and a fourth set of detectors 18D may be positioned along the second axis 276 and may form a fourth sensing unit 280 for obtaining a fourth regional saturation measurement. As shown, the third set of detectors 18C may be positioned on one side of a first axis 270, while the fourth set of detectors 18D may be positioned on another side of the first axis 270.

The signals received from the detectors 18 of the sensor 12 of FIG. 12 and/or the regional oxygen saturation measurements determined based on the received signals, may be processed and/or displayed in a similar manner as disclosed above with respect to FIG. 3. For example, the four regional saturation measurements determined based on the signals received from the four sensing units 272, 274, 278, 280 may be compared, averaged, and/or displayed. The monitor 14 may be configured to evaluate uniformity of the tissue underlying the sensor 12 based on a variance between the signals and/or measurements, for example. As discussed above, additional emitters 16 may be used to determine additional information related to uniformity of the tissue. In some embodiments, the monitor 14 may be configured to determine whether the sensor 12 should be repositioned based on the variance, as set forth above. As compared with the sensor 12 of FIG. 3, the configuration of the sensor 12 in FIG. 12 may be desirable for certain monitoring sites and/or may provide more signals and measurements that can be utilized in the above-described processing steps, for example.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A medical sensor, comprising:
a substrate;
a first set of optical components configured to obtain a first set of signals for determining a first regional oxygen saturation measurement, comprising:
a first emitter disposed on the substrate;
a first detector disposed on the substrate, the first detector being separated from the first emitter by a first distance along a first axis; and
a second detector disposed on the substrate, the second detector being separated from the first emitter by a second distance along the first axis, wherein the second distance is greater than the first distance; and
a second set of optical components configured to obtain a second set of signals for determining a second regional oxygen saturation measurement, comprising:
a second emitter disposed on the substrate; and
a third detector disposed on the substrate, the third detector being separated from the second emitter by a third distance along a second axis different from the first axis.

2. The medical sensor of claim 1, wherein the second set of optical components comprises the first detector.

3. The medical sensor of claim 2, wherein the first detector is separated from the second emitter by a fourth distance along the second axis, wherein the fourth distance is approximately equal to the first distance and is less than the third distance.

4. The medical sensor of claim 3, wherein a two-dimensional monitoring area on a patient defined by the first emitter, the second emitter, the second detector, and the third detector when the medical sensor is disposed on the patient is at least 5 cm².

5. The medical sensor of claim 1, wherein the first distance is between about 10 mm and 20 mm and the second and the third distances are between about 30 mm and 40 mm.

6. The medical sensor of claim 1, comprising:
a third set of optical components configured to obtain a third regional oxygen saturation measurement, comprising:
the first emitter;
a fourth detector and a fifth detector separated from the first emitter along a third axis;
a fourth set of optical components configured to obtain a fourth regional oxygen saturation measurement, comprising:
the second emitter; and
the fourth detector and a sixth detector separated from the second emitter along a fourth axis.

7. The medical sensor of claim 6, wherein a two-dimensional monitoring area on a patient defined by the second detector, the third detector, the fifth detector, and the sixth detector when the medical sensor is disposed on the patient is at least 5 cm².

8. The medical sensor of claim 1, comprising a third set of optical components configured to obtain a third regional oxygen saturation measurement, comprising:
the second detector;
the third detector; and
a third emitter disposed on the substrate and separated from the second detector and the third detector along a third axis different from the first and second axes.

9. The medical sensor of claim 8, comprising a fourth set of optical components configured to obtain a fourth regional saturation measurement, comprising:
the second detector;
the third detector; and
a fourth emitter disposed on the substrate and separated from the second detector and the third detector and the third emitter along the third axis.

10. The medical sensor of claim 1, comprising an axis of symmetry extending through the first detector, wherein the first and second sets of optical components are arranged symmetrically about the axis of symmetry.

11. The medical sensor of claim 1, wherein the first emitter is configured to emit wavelengths of light different than wavelengths of light emitted by the second emitter.

12. A medical monitoring system, comprising:
a medical sensor, comprising:
a substrate;
a first set of optical components disposed on the substrate and configured to obtain a first set of signals for determining a first regional oxygen saturation measurement comprising:
a first emitter spaced apart from a first detector and a second detector along a first axis;
a second set of optical components disposed on the substrate and configured to obtain a second set of signals for determining a second regional oxygen saturation measurement, comprising:
a second emitter disposed spaced apart from the first detector and a third detector along a second axis different from the first axis; and
a medical monitor configured to:
receive the first and second sets of signals; and
determine a first regional oxygen saturation measurement based on the first set of signals and a second regional oxygen saturation measurement based on the second set of signals.

13. The medical monitoring system of claim 12, wherein the medical monitor is configured to provide a notification to reposition the medical sensor on a patient if the first and second sets of signals differ by more than a predetermined threshold amount.

14. The medical monitoring system of claim 12, wherein the medical sensor comprises an axis of symmetry extending between the first detector, wherein the first and second sets of optical components are arranged symmetrically about the axis of symmetry.

15. The medical monitoring system of claim 11, wherein the first emitter is configured to emit wavelengths of light different than wavelengths of light emitted by the second emitter.

16. The medical monitoring system of claim 11, wherein the first and second emitters are aligned along a first vertical axis of the medical sensor and wherein the second and third detectors are aligned along a second vertical axis of the medical sensor parallel to the first vertical axis.

17. The medical monitoring system of claim 16, comprising a third emitter aligned with the first and second emitters along the first vertical axis and a fourth detector aligned with the second and third detectors along the second vertical axis, wherein the third emitter, first detector, and fourth detector form a third set of optical components configured to obtain a third set of signals for determining a third regional oxygen saturation measurement.

18. The medical monitoring system of claim 17, wherein the medical monitor is configured to determine the first, second, and third regional oxygen saturation measurements using the first, second, and third sets of optical components, respectively, to determine a mean of the measurements, and to exclude one or more measurements that vary by more than a predetermined difference from the mean.

19. A medical monitoring system, comprising:
a medical sensor, comprising:
a first set of optical components configured to obtain a first set of signals for determining a first regional oxygen saturation measurement comprising:
a first emitter configured to emit a first wavelength and a third wavelength of light; and
a second set of optical components comprising configured to obtain a second set of signals for determining a second regional oxygen saturation measurement comprising:
a second emitter configured to emit a second wavelength and a fourth wavelength of light, wherein the first wavelength is less than the second wavelength, which is less than the third wavelength, which is less than the fourth wavelength; and
a medical monitor configured to:
receive the first and the second sets of signals; and
determine a first regional oxygen saturation measurement based on the first set of signals and a second regional oxygen saturation measurement based on the second set of signals.

20. The medical sensor of claim 19, wherein the medical sensor comprises two or more detectors spaced apart from the first emitter and the second emitter linearly along an axis of the medical sensor.
